(19) United States
(12) Patent Application Publication
(10) Pub. No.: US 2011/0137152 A1
(43) Pub. Date: Jun. 9, 2011

(54) SYSTEM AND METHOD FOR COOLING COMPONENTS OF A SURGICAL NAVIGATION SYSTEM

(75) Inventor: Dun Alex Li, Salem, NH (US)

(73) Assignee: General Electric Company, Schenectady, NY (US)

(21) Appl. No.: 12/630,551

(22) Filed: Dec. 3, 2009

(51) Int. Cl. A61B 5/05

(52) U.S. Cl. 600/424

(57) ABSTRACT

In one embodiment, a surgical navigation system is provided with a transmitter assembly that includes at least one coil for creating an electromagnetic field in the vicinity of a surgical operation. The surgical navigation system also includes a cooling unit thermally coupled to the transmitter assembly for dissipating heat generated by the transmitter assembly and a receiver assembly that includes at least one coil configured to generate signals induced by the field. The surgical navigation system further includes electronic circuitry coupled to and communicating with the transmitter assembly and the receiver assembly for calculating at least a position of the receiver coil.

### BACKGROUND OF THE INVENTION

The subject matter disclosed herein relates generally to a surgical navigation system that uses electromagnetic fields to determine the position of an object, and more particularly to a system and method for cooling components of the surgical navigation system.

In medical applications, surgical navigation systems have been used to provide an operator (e.g. a physician, surgeon, or other medical practitioner) with information to assist in the precise and rapid positioning of a medical device or instrument located in or near a patient's body during image-guided surgery. An electromagnetic tracking system provides positioning and orientation information for a medical device or instrument with respect to the patient or a reference coordinate system. When integrated or coordinated with an imaging system, such electromagnetic tracking systems may provide intraoperative tracking of the precise location of a medical device or instrument in relation to multidimensional images of a patient's anatomy.

In image-guided systems, a surgical navigation system uses visualization tools to provide a medical practitioner with co-registered views of a graphical representation of the medical device or instrument with pre-operative or intraoperative images of the patient's anatomy. In other words, a surgical navigation system allows a medical practitioner to visualize the patient's anatomy, and a displayed image may be continuously or periodically updated to reflect the real-time position and orientation of the medical device or instrument. The combination of the image and the representation of the tracked medical device or instrument provide position and orientation information that allows a medical practitioner to manipulate the medical device or instrument to a desired location with an accurate position and orientation.

Generally, electromagnetic tracking systems include electromagnetic transmitters and electromagnetic receivers with at least one coil or a coil array. An alternating drive current signal is provided to each coil in the electromagnetic transmitter, generating an electromagnetic field emitted from the coils. The field induces a current in each coil of the receiver. These signals are detected and sent to a computer for processing. The computer uses these measured signals to calculate the position and orientation of the coils of the receiver relative to the transmitter, in some systems, including six degrees of freedom (x, y, and z measurements, as well as roll, pitch and yaw angles).

Preferably, the mutual inductances between coils of the transmitter and the receiver may be controlled and measured without inaccuracies. However, electromagnetic tracking systems are known to suffer from accuracy degradation due to such factors as electromagnetic field distortion, such as may be caused by the presence of an uncharacterized metal distorter within the tracking volume or electromagnetic fields of the electromagnetic tracking system. To improve tracking accuracy and tolerance to such perturbations, electromagnetic tracking systems may employ low frequency tracking. However, low frequency tracking may result in a reduced tracking volume. To compensate for this effect, the power input to the transmitter coils to drive the field may be increased. Unfortunately, increasing the power may result in increased heating of the electromagnetic transmitter which may produce tracking inaccuracies and adversely affect other system components. This also represents a source of heat in what is generally an otherwise temperature controlled environment, possibly leading to discomfort for the patient and attending physicians and technicians.

Therefore, there is a need for a system and method to cool components of an electromagnetic tracking system within a surgical navigation system.

### BRIEF DESCRIPTION OF THE INVENTION

In accordance with one embodiment, a surgical navigation system is provided with a transmitter assembly that includes at least one coil for creating an electromagnetic field in the vicinity of a surgical operation. The surgical navigation system also includes a cooling unit thermally coupled to the transmitter assembly for dissipating heat generated by the transmitter assembly, and a receiver assembly that includes at least one coil configured to generate signals induced by the field. The surgical navigation system further includes electronic circuitry coupled to and communicating with the transmitter assembly and the receiver assembly for calculating at least a position of the receiver coil.

In accordance with another embodiment, a surgical navigation system is provided with an imaging system operable to acquire image data, a display configured to display an image acquired via the imaging system, and an electromagnetic tracking system. The electromagnetic tracking system includes at least one transmitter assembly located beneath an imaged subject and configured to generate an electromagnetic field, at least one receiver assembly configured to generate signals induced by the electromagnetic field, and electronics coupled to and communicating with the transmitter assembly and the receiver assembly for calculating at least a position of the receiver assembly. The surgical navigation system also includes a cooling system thermally coupled to the transmitter assembly to dissipate heat generated by the transmitter assembly.

In accordance with a further embodiment, a method of cooling a transmitter assembly of a surgical navigation system is provided. The method provides for measuring a temperature of a transmitter assembly, communicating the temperature of the transmitter assembly to a controller, and activating a cooling system to dissipate heat generated by the transmitter assembly and to reduce the temperature of the transmitter assembly.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a schematic representation of a surgical navigation system in accordance with an embodiment of the invention;

FIG. 2 is a diagrammatical perspective view of a transmitter assembly for a system of the type shown in FIG. 1;

FIG. 3 is a diagrammatical perspective view of an alternative transmitter assembly for a system of the type shown in FIG. 1;

FIG. 4 is a diagrammatical perspective view of a transmitter assembly secured within a surgical table;

FIG. 5 is a diagrammatical side view of a transmitter assembly secured within a surgical table with a passive cooling system;

FIG. 6 is a diagrammatical side view of a transmitter assembly secured within a surgical table with an active cooling system;

FIG. 7 is a diagrammatical side view of a transmitter assembly secured within a surgical table with an alternative active cooling system;

FIG. 8 is a diagrammatical side view of a transmitter assembly secured within a surgical table with a further alternative active cooling system;

FIG. 9 is a schematic diagram of the thermoelectric cooling system shown in FIG. 8;

FIG. 10 is a block diagram of a transmitter assembly regulated in a closed-loop manner;

FIG. 11 is a block diagram of a transmitter assembly regulated in an open loop manner;

FIG. 12 is a flow diagram of a method to cool a component of a surgical navigation system; and

FIG. 13 is a flow diagram of a method to cool a component of a surgical navigation system.

### DETAILED DESCRIPTION OF THE INVENTION

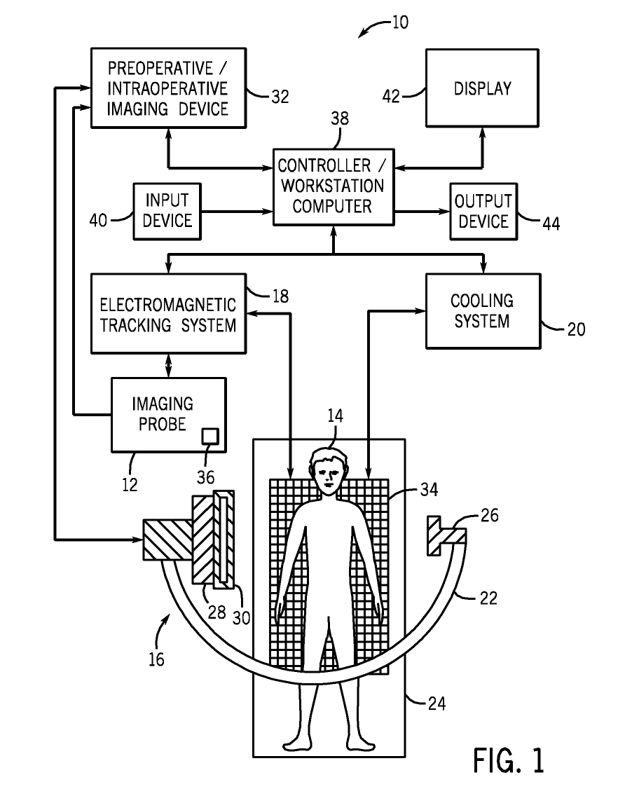

Turning now to the drawings, FIG. 1 illustrates an exemplary embodiment of a surgical navigation system 10 operable to track movement of a tool or object 12 (e.g., imaging probe or catheter) through an anatomy of an imaged subject 14. The system generally includes an imaging system 16, an electromagnetic tracking system 18 operable to track movement of a tool or object 12 through the imaged subject 14, and a cooling system 20 to cool components of the electromagnetic tracking system 18.

The imaging system 16 is generally operable to generate a two-dimensional, three-dimensional, or four-dimensional image data corresponding to an area of interest of the imaged subject 14. Examples of the imaging system 16 can include, but are not limited to, computed tomography (CT), magnetic resonance imaging (MRI), x-ray or radiation, positron emission tomography (PET), computerized tomosynthesis, ultrasound (US), angiographic, fluoroscopic, and the like or combination thereof. The imaging system 16 can be operable to generate static images acquired by imaging detectors prior to a medical procedure, or real-time or near real-time images acquired during the progress of a procedure (e.g., angioplastic procedures, laparoscopic procedures, endoscopic procedures, etc.). Thus, the type of images can be diagnostic or interventional.

The illustrated imaging system 16 includes a conventional C-arm 22 positioned to direct a radiation beam at an imaged subject 14 positioned on a surgical table 24. The imaging system further includes a radiation source 26 and an image intensifier 28. A navigation calibration target 30 may be rigidly attached to the image intensifier 28. If utilized, the calibration target 30 may comprise an array of radio-opaque fiducials and a plurality of electromagnetic sensors. The calibration target 30 may be coupled to the electromagnetic tracking system 18 via a cable. The imaging system 16 may also be coupled to a preoperative and/or intraoperative imaging device 32. The radiation source 26 and image detector 28 of the imaging system 16 may be selectively moved to various positions so as to acquire image data (e.g., two-dimensional, three-dimensional) at different views of one or more regions of interest of the medical imaged subject 14, or four-dimensional data (three-dimensional data over a desired time period).

The navigation portion of the surgical navigation system 10 includes an electromagnetic tracking system 18. The electromagnetic tracking system 18 includes at least one transmitter assembly 34 including at least one coil located near, such as beneath, the imaged subject 14 and at least one receiver assembly 36 including at least one coil located within a tool or object 12. Alternatively, the at least one receiver assembly 36 may be rigidly attached to an internal organ or to the external body of the imaged subject 14 in a conventional manner. For purposes of the disclosure, the tool or object 12 is defined to include any flexible medical delivery system such as, for example, an endoscope or a catheter. The electromagnetic tracking system 18 further includes electronics coupled to and communicating with both the transmitter assembly 34 and the receiver assembly 36 to determine or calculate the position and orientation of the receiver coil within the receiver assembly 36.

The transmitter assembly 34 may be a wireless or wired device. The receiver assembly 36 may also be a wireless or wired device. In embodiments employing a wireless transmitter assembly 34 or wireless receiver assembly, separate power units may be provided, such as batteries or photocells, for example.

The electromagnetic tracking system 18 may include drive circuitry configured to provide a drive current to each coil of the at least one transmitter assembly 34. By way of example, a drive current may be supplied by the drive circuitry to energize a coil or coils of the transmitter assembly 34, and thereby generate an electromagnetic field that is detected by a coil of the receiver assembly 36. The drive current may comprise a periodic waveform with a given frequency (e.g., a sinusoidal or other periodic signal). The drive current supplied to the transmitter coils will generate an electromagnetic field at the same frequency as the drive current. The electromagnetic field then induces a current indicative of the mutual inductance in the coils of the receiver assembly 36. The electromagnetic tracking system 18 includes receiver data acquisition circuitry for receiving signals from the coil or coils if the receiver, the signals being representative of the induced current (or a corresponding measured voltage).

The surgical navigation system 10 may further include a controller or workstation computer 38 coupled to and receiving data from the receiver assembly 36. The same (or a different) controller may also receive signals or data from the transmitter assembly, such as temperature-related data as discussed below. The controller 38 is generally operable to register the position and orientation information of the transmitter assembly 34 or receiver assembly 36 relative to the acquired imaging data from the imaging system 16. System 10 is thereby operable to determine the location (and if appropriately configured, the orientation) of the receiver assembly 36 or attached tool 12 relative to the transmitter assembly field, and to correlate this location (and orientation) to one or more pre-acquired or real-time images acquired by the imaging system 16.

The controller or workstation computer 38 is generally connected and in communication with and controls the imaging system 16, the preoperative and/or intraoperative imaging device 32, the electromagnetic tracking system 18, and the cooling system 20 so as to enable each to be in synchronization with one another and to enable the data acquired therefrom to produce or generate a full-view model of the imaged subject 14. The controller 38 includes at least one processor and memory circuitry. The processor can be arranged independent of or integrated with the memory. Although the processor and memory are described in the controller 38, it should be understood that the processor or memory or portions thereof can be located at the imaging system 16, the electromagnetic tracking system 18, or cooling system 20 or combination thereof. The processor is generally operable to execute program instructions stored within the memory such as any algorithms to use the measured signals indicative of the mutual inductance to calculate the position (and orientation) of the receiver assembly 36 relative to the transmitter assembly 34, or vice versa. The processor can also be capable of receiving input or information or communicating output data. Examples of the processor include a digital signal processor, a central processing unit, or the like.

An embodiment of the memory generally comprises one or more computer-readable media operable to store a plurality of computer-readable program instructions for execution by the processor. The memory can also be operable to store data generated or received by the controller 38. By way of example, such media may include RAM, ROM, PROM, EPROM, EEPROM, flash, CD-ROM, DVD, or other known computer-readable media or combinations thereof which can be used to carry or store desired program code in the form of instructions or data structures and which can be accessed by a general purpose or special purpose computer or other machine with a processor.

The controller 38 further includes or is in communication with an input device 40, a display 42, and an output device 44. The input device 40 can be generally operable to receive and communicate information or data from a user to the controller 38. The input device 40 can include a mouse device, pointer, keyboard, touch screen, microphone, or other like device or combinations thereof capable of receiving a user directive. The display 42 is generally operable to illustrate output data for viewing by the user. An embodiment of the display 42 can be operable to simultaneously illustrate or fuse static or real-time image data generated by the imaging system 16 with tracking data generated by the electromagnetic tracking system 18. The display 42 is capable of illustrating two-dimensional, three dimensional, and/or four-dimensional image data or a combination thereof through shading, coloring, and/or the like. Examples of the display 42 include a cathode ray monitor, a liquid crystal display (LCD) monitor, a touchscreen monitor, a plasma monitor, or the like or combination thereof. The output device 44 can be generally operable to illustrate or audibilize output data for viewing or for listening, respectively, by the user. The output device 44 can include a display, a visual alarm, an audible alarm, or other like device or combination thereof.

Each of the transmitter coil and the receiver coil is operable to generate and/or sense an electromagnetic field. Structurally, the coils of the transmitter and receiver assemblies 34 and 36 may be built with various coil architectures. The one or more coils of the transmitter assembly 34 may be single coils, a pair of single coils, industry-standard-coil architecture (ISCA) type coils, a pair of ISCA type coils, multiple coils, or an array of coils. The one or more coils of the receiver assembly 36 may be single coils, a pair of single coils, ISCA type coils, a pair of ISCA type coils, multiple coils, or an array of coils.

ISCA type coils are defined as three approximately collocated, approximately orthogonal, and approximately dipole coils. Therefore, ISCA transmitter and receiver coils would include three approximately collocated, approximately orthogonal, and approximately dipole coils for the transmitter assembly 34 and three approximately collocated, approximately orthogonal, and approximately dipole coils for the receiver assembly 36. In other words, an ISCA configuration for the transmitter and receiver assemblies 34 and 36 would include a three-axis dipole coil transmitter and a three-axis dipole receiver. In the ISCA configuration, the transmitter coils and the receiver coils are configured such that the three coils (i.e., coil trios) exhibit the same effective area, are orientated orthogonally to one another, and are centered at the same point.

In one embodiment, the coils of the at least one transmitter assembly 34 may be characterized as single dipole coils and emit magnetic fields when a current is passed through the coils. Those skilled in the art will appreciate that multiple electromagnetic field generating coils may be used in coordination to generate multiple magnetic fields. Similar to the at least one transmitter assembly 34, the coils of the at least one receiver assembly 36 may be characterized as single dipole coils and detect the magnetic fields emitted by the at least one transmitter assembly 34. When a current is applied to the coils of the transmitter assembly 34, a field is created that will include the patient and the vicinity in which the receiver assembly will be used. This field, then, induces a current in each coil of the receiver assembly 36, which may be measured, sensed, or otherwise detected to generate an output signal for analysis.

The operating environment of the imaging probe 12 may comprise various objects, or sources of field perturbation, sometimes referred to as distorters, which may influence the quality or uniformity of the field, and thus the quality of the sensed signals from the receiver coil, thereby reducing the tracking accuracy. Using low frequency tracking (e.g., a 10 to 20 kHz carrier frequency in the transmitter assembly 34) may help in reducing the effect of distortion in tracking the receiver coil. While low frequency tracking may increase tracking accuracy, the tracking volume or tracking range may be reduced. In order to improve the tracking volume/range, the power to the transmitter assembly 34 may be increased. However, the increased power may increase the temperature of the transmitter assembly 34 (which could itself adversely affect tracking accuracy).

The surgical navigation system 10 includes a cooling system 20 thermally coupled to the transmitter assembly 34 to dissipate heat generated by the transmitter assembly 34. The cooling system 20 may be coupled to a controller 38 configured to regulate the operation of the cooling system 20. The cooling system 20 may be integrated into the transmitter assembly 34, or provided as a separate system or component, as described below.

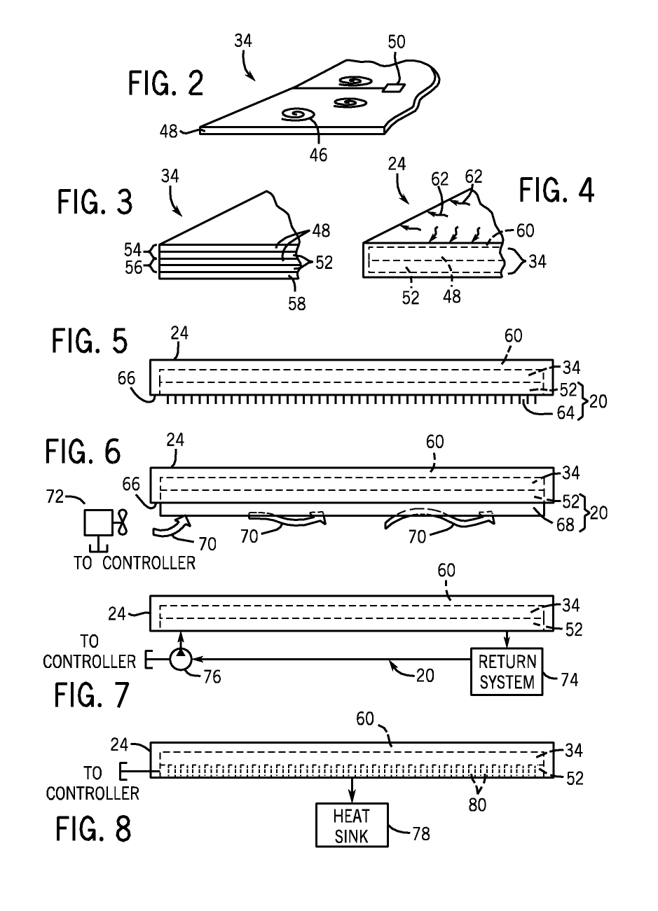

FIG. 2 illustrates an embodiment of a transmitter assembly 34 employed in the surgical navigation system 10, which in this embodiment is formed by a plurality of flat coils of curved conductor traces forming spiral coils 46 on one or more printed circuit boards (PCBs) 48. Various shapes, forms and configurations of such coils may be envisioned, with the exemplary spiral pattern being shown only by way of example. The coils 46 are preferably copper traces with spaces between adjacent coils. The coils may be provided on one or both sides of a PCB 48. The PCB 48 may also include multiple layers with coils disposed in the various layers. The PCB 48 may be made of a material that is rigid or flexible. The coils 46 are arranged to generate electromagnetic fields and gradients in three axes (x, y, z directions) above the assembly. The x and y directions are in the plane of the assembly. The z direction is perpendicular to the plane of the assembly.

The cooling system 20 may further include one or more temperature sensors or indicators 50 thermally coupled to the transmitter assembly 34. The temperature sensor 50 may also be electronically coupled to and communicating with the controller 38. The controller 38 may be configured to regulate the temperature of the transmitter assembly 34 in a closed loop manner based upon sensed temperature, as described below, or may simply use sensed temperature data to provide an indication to the system user.

FIG. 3 illustrates another embodiment of the transmitter assembly 34. The transmitter assembly 34 may include a first layer of PCB 48 that includes at least one coil (coils not shown) and a second layer that includes the cooling unit 52. The illustrated transmitter assembly 34 includes a first region 54 that includes a PCB layer 48 that includes at least one coil and a cooling unit layer 52, followed by a second region 56 that includes both a PCB layer 48 and cooling unit layer 52. In some embodiments, the transmitter assembly 34 may include an electromagnetic shielding layer 58 to reduce the influence of external electromagnetic fields or the cooling system 20 on the fields generated by the transmitter assembly 34.

FIG. 4 illustrates a further embodiment where the transmitter assembly 34 is secured within the surgical table 24. In some embodiments, the transmitter assembly 34 may be coupled to the surgical table 24. Additionally, the transmitter assembly 34 may be secured within the surgical table 24 beneath a patient support 60. The transmitter assembly 34 secured within the surgical table 24 may include at least a first layer of PCB 48 that includes at least one coil and a second layer that includes a cooling unit 52. The cooling system 20 may dissipate the heat generated by the transmitter assembly 34 away from the transmitter assembly 34 to the outer edges of the surgical table 24.

FIG. 5 illustrates an embodiment of a transmitter assembly 34 secured within the surgical table 24 with a passive cooling system 20. The transmitter assembly 34 and the cooling unit layer 52 are secured beneath the patient support 60. The cooling unit layer 52 may include extensions or fins 64 extending below the bottom 66 of the surgical table 24 to act as a heat sink and to form a passive cooling system 20. The cooling unit layer 52 transfers heat away from the transmitter assembly 32 to the extensions or fins 64 below the bottom 66 of the surgical table 24 to allow air to convectively cool the extensions or fins 64. The extensions or fins 64 may vary in number and size, as well as in shape.

FIG. 6 illustrates an embodiment of a transmitter assembly 34 secured within the surgical table 24 with an active cooling system 20. The transmitter assembly 34 and the cooling unit layer 52 are secured beneath the patient support 60. The cooling unit layer 52 may include a portion 68 extending below the bottom 66 of the surgical table 24 to form an active cooling system 20. The cooling unit layer 52 transfers heat away from the transmitter assembly 34 to the portion 68 extending below the bottom 66 of the surgical table 24 to act as a heat sink and to allow air 70 flowing from a fan 72 to cool the transmitter assembly 34. The fan 72 may be coupled to a controller 38. The fan 72 may be controlled in an open or closed-loop manner. A user may operate the fan 72 via the controller 38. Alternatively, the controller 38 may receive data related to the temperature of the transmitter assembly 34 to allow the controller 38 to regulate operation of the fan 72 without user input (e.g., turn on or off, or alter the speed).

FIG. 7 illustrates another embodiment of a transmitter assembly 34 secured within the surgical table 24 with an active cooling system 20. Similar to FIGS. 4-6, the transmitter assembly 34 and the cooling unit layer 52 are secured beneath the patient support 60. The active cooling system 20 may include the cooling unit layer 52 coupled to a return system 20, the return system coupled to a pump 76, and the pump 76 coupled to the controller 38. In one embodiment, the cooling unit layer 52 may have water or another cooling fluid running throughout the layer 52 to cool the transmitter assembly 34. The heat from the transmitter assembly 34 may be transferred to the water and the pump 76 may drive the cooling fluid from the cooling unit layer 52 to the return system 74. The return system 74 may include a filtration system, a fluid reservoir, a heat sink to receive the heat from the cooling fluid. The pump 76 may then draw the fluid from the return system 74 and recirculate it back to the cooling unit layer 52. The controller 38 may regulate the pump 76 in an open or closed-loop fashion.

FIG. 8 illustrates a further embodiment of a transmitter assembly 34 secured within the surgical table 24 with an active cooling system 20. Similar to FIGS. 4-7, the transmitter assembly 34 and the cooling unit layer 52 are secured beneath the patient support 60. The active cooling system 20 may employ a thermoelectric cooling system. The cooling system 20 may include the cooling unit layer 52 coupled to a heat sink 78. The cooling unit may include a matrix of semiconductor thermocouples 80. The semiconductor thermocouples may be coupled to the transmitter assembly 34 to dissipate heat from the transmitter assembly to the heat sink 78. The controller 38 may be coupled to semiconductor thermocouples 80 and regulate the cooling system 20 in an open or closed-loop fashion.

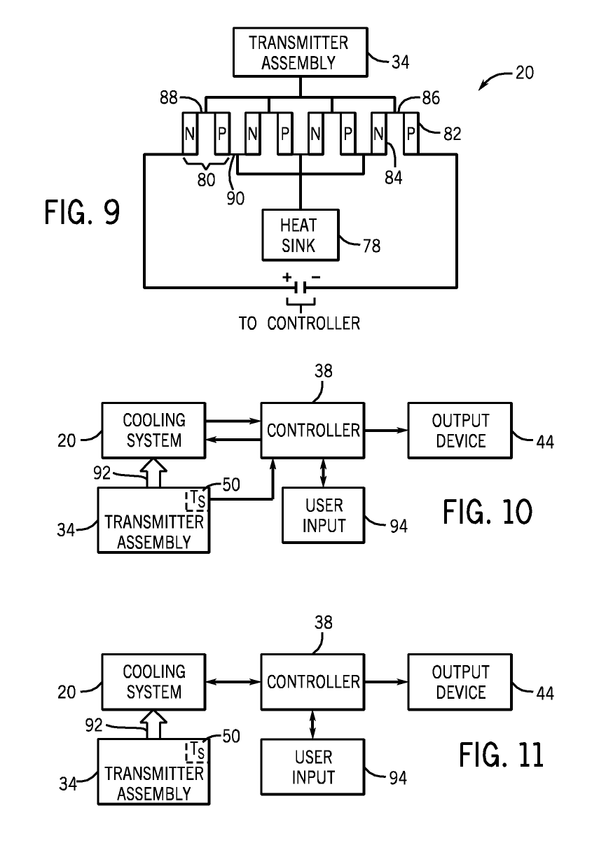

FIG. 9 illustrates an embodiment of the thermoelectric cooling system 20 of FIG. 8. The thermoelectric cooling system 20 includes a plurality of semiconductor thermocouples 80 and a controller 38 coupled to the plurality of semiconductor thermocouples 80. The semiconductor thermocouple 80 may be a P-N type semiconductor thermocouple consisting of a P-type semiconductor material (Bi2Te3-Sb2Te3) 82 and N-type semiconductor material (Bi2Te3-Bi2Te3) 84. The semiconductor material of the semiconductor thermocouples 80 may vary. The P-type and N-type semiconductors 82 and 84 are connected at one end by a metallic connection 86. The opposing end of the P-type semiconductor material 82 is connected to the negative pole (−) of the power supplied by the controller 38 and the N-type semiconductor material 84 is connected to the positive pole (+) of the power supplied by the controller 38. The plurality of semiconductor thermocouples 80 are alternatively coupled at an upper end 88 or a lower end 90 to form a thermoelectric cooling system 20 using the Peltier effect. The upper ends 88 are connected and thermally coupled to the transmitter assembly 34 and the lower ends 90 to a heat sink 78. Current flows from the N-type semiconductor material 84 to the P-type semiconductor material 82, causing an endothermic effect at the upper end 88. A corresponding exothermic reaction occurs at the opposing lower end 90 of the N-type and P-type semiconductor materials 84 and 82. In cumulative combination, heat is transferred from the transmitter assembly 34 to the semiconductor thermocouples 80, then from the upper ends (endothermic ends) 88 of the semiconductor thermocouples 80 to the lower ends (exothermic ends) 90, where the heat may then be transferred to the heat sink 78 and dissipated. The cooling capacity of the thermoelectric cooling system 20 may vary based on combining differing numbers of semiconductor thermocouples 80 electrically in series.

As mentioned above, the temperature of the transmitter assembly 34 may be regulated in an open or closed-loop manner. FIGS. 10-11 illustrate a transmitter assembly 34 regulated in a closed and an open loop manner, respectively. In one embodiment, a surgical navigation system 10 may include a transmitter assembly 34 that may include a temperature sensor or indicator 50, a cooling system 20 to dissipate the heat 92 generated by the transmitter assembly 34, and a controller/workstation computer 38 configured to receive user input 94 connected to an output device 44. The temperature sensor 50 may be thermally coupled to the controller 38. In addition, the temperature sensor 50 may be electronically coupled to and communicating with the controller 38. The temperature sensor 50 may also be coupled to circuitry that includes an output device 44 configured to indicate the temperature of the transmitter assembly 34. The output device 44 may be separate from the controller 38 or integrated into the controller 38. In some embodiments, the output device 44 may be a display to indicate the numerical value of the temperature of the transmitter assembly 34. In other embodiments, the output device 44 may provide a color indicator for the temperature of the transmitter assembly 34. Further embodiments, the output device 44 may include an audible alarm to indicate the temperature of the transmitter assembly 34 is above a certain threshold level.

As shown in FIG. 10, the controller 38 may be configured for regulating the temperature of the transmitter assembly 34 in a closed-loop manner. The controller 38 may receive user input 92 from a user setting a specific temperature for the transmitter assembly 34 to be maintained at by the cooling system 20. Alternatively, the controller 38 may regulate the cooling system 20 to keep the transmitter assembly 34 at or near a default setting. The controller 38 may directly regulate the temperature of the transmitter assembly 34 via the activation or deactivation of the cooling system 20.

In the open loop regulation of the transmitter assembly temperature, shown in FIG. 11, the user may in response to data received from the output device 44 activate the cooling system 20 via user input 94 entered into the controller 38. Once the transmitter assembly 34 reaches the desired temperature the user may deactivate the cooling system 20 via user input 94 entered into the controller 38.

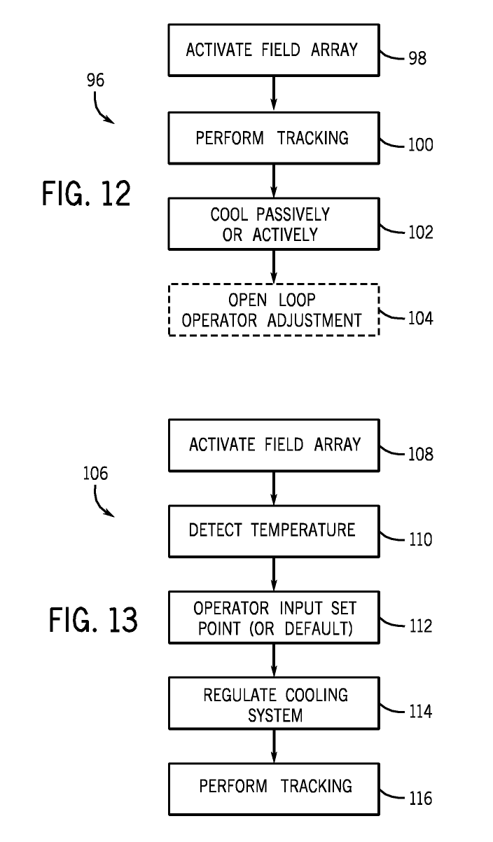

FIG. 12 is a flow diagram depicting an embodiment of a method 96 for cooling a component of a surgical navigation system. The method 96 may be performed on a surgical navigation system 10 including an electromagnetic tracking system 18 that includes at least one transmitter assembly 34 and at least one receiver assembly 36, a cooling system 20, and a controller 38. The method may be performed by at least one computer program or algorithm running on a controller or workstation computer 38.

The method comprises activating a field array, as indicated at step 98, an electromagnetic field being generated by the field array, such as the transmitter assembly 34 described above. Tracking is then performed at step 100, as discussed above. In particular, the electromagnetic tracking system 18 tracks the receiver assembly relative to the transmitter assembly 34. The transmitter assembly 34 is then cooled passively or actively, as indicated at step 102. The method 96 may continue at step 104 with an open loop operator adjustment either to continue to cool the transmitter assembly 34 or to cease cooling.

FIG. 13 is a flow diagram illustrating an embodiment of a method 106 for closed loop cooling of a component of a surgical navigation system 10. As illustrated, the method comprises activating a field array at step 108, such as via the transmitter assembly 34 described above. At step 110, the temperature of the assembly 34 (or of a component associated with or near the assembly 34) is detected, such as via a temperature sensor 50 that provides a temperature signal to a controller 38 as discussed above. An operator input set point (or default) may be detected, as indicated at step 112, where an operator or user provides a temperature that the transmitter assembly 34 should maintain (or alternatively the controller 38 may use a default temperature). The cooling system 20 is then regulated as indicated at step 114, as described above. The system 18 may, during this time, performing tracking, as indicated at step 116.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A surgical navigation system comprising:
a transmitter assembly comprising at least one coil for creating an electromagnetic field in the vicinity of a surgical operation;
a cooling unit thermally coupled to the transmitter assembly for dissipating heat generated by the transmitter assembly;
a receiver assembly comprising at least one coil configured to generate signals induced by the field; and
electronic circuitry coupled to and communicating with the transmitter assembly and the receiver assembly for calculating at least a position of the receiver coil.

2. The surgical navigation system of claim 1, wherein the cooling unit comprises a plurality of semiconductor thermocouples.

3. The surgical navigation system of claim 2, wherein the semiconductor thermocouples are powered by a controller to generate both an endothermic end and an exothermic end on each of the semiconductor thermocouples.

4. The surgical navigation system of claim 3, wherein the endothermic ends of the semiconductor thermocouples are connected to the transmitter assembly and the exothermic ends of the semiconductor thermocouples are connected to a heat sink.

5. The surgical navigation system of claim 1, wherein the cooling unit comprises a heat sink that is air cooled or water-cooled.

6. The surgical navigation system of claim 1, further comprising a temperature sensor.

7. The surgical navigation system of claim 6, wherein the temperature sensor is electronically coupled to and communicating with a controller configured for regulating the temperature of the transmitter assembly.

8. The surgical navigation system of claim 7, wherein the controller is configured to regulate the temperature of the transmitter assembly independent of user input.

9. The surgical navigation system of claim 6, wherein the temperature sensor is coupled to circuitry comprising an output device configured to indicate the temperature of the transmitter assembly.

10. The surgical navigation system of claim 1, wherein the transmitter coil and the receiver coil are both operable to generate and sense an electromagnetic field.

11. The surgical navigation system of claim 1, wherein the transmitter assembly is coupled to a surgical table.

12. The surgical navigation system of claim 11, wherein the transmitter assembly is secured within the surgical table beneath a patient support.

13. The surgical navigation system of claim 11, wherein heat generated by the transmitter assembly is dissipated away from the transmitter assembly to outer edges of the surgical table.

14. The surgical navigation system of claim 1, wherein the transmitter assembly comprises a first layer that includes the at least one coil and a second layer that includes the cooling unit.

15. The surgical navigation system of claim 1, wherein the transmitter assembly comprises an electromagnetic shielding layer.

16. A surgical navigation system comprising:
an imaging system operable to acquire image data;
a display configured to display an image acquired via the imaging system;
an electromagnetic tracking system comprising:
at least one transmitter assembly located beneath an imaged subject and configured to generate an electromagnetic field;
at least one receiver assembly configured to generate signals induced by the electromagnetic field; and
electronics coupled to and communicating with the transmitter assembly and the receiver assembly for calculating at least a position of the receiver assembly; and
a cooling system thermally coupled to the transmitter assembly to dissipate heat generated by the transmitter assembly.

17. The surgical navigation system of claim 16, comprising a controller coupled to the cooling system and configured to regulate operation of the cooling system.

18. The surgical navigation system of claim 17, wherein the cooling system comprises a temperature sensor, and wherein the controller is coupled to the temperature sensor and controls operation of the cooling system in a closed-loop manner.

19. The surgical navigation system of claim 16, wherein the cooling system comprises a plurality of semiconductor thermocouples and a controller coupled to the plurality of semiconductor thermocouples to drive each of the semiconductor thermocouples to extract heat from the at least one transmitter assembly.

20. The surgical navigation system of claim 16, wherein the transmitter assembly comprises a temperature sensor electronically coupled to and communicating with the controller, and wherein the controller is configured for regulating the temperature of the transmitter assembly.

21. The surgical navigation system of claim 20, wherein the controller is coupled to a temperature indicator, and wherein the display is configured to indicate a status of the transmitter assembly temperature.

22. The surgical navigation system of claim 16, wherein the controller is configured to reduce the temperature of the transmitter assembly independent of user input.

23. A method of cooling a transmitter assembly of a surgical navigation system, the method comprising:
measuring a temperature of a transmitter assembly;
communicating the temperature of the transmitter assembly to a controller; and
activating a cooling system to dissipate heat generated by the transmitter assembly and to reduce the temperature of the transmitter assembly.

24. The method of claim 23, wherein the cooling system is activated by the controller and/or a user.

25. The method of claim 23, wherein the cooling system comprises a plurality of semiconductor thermocouples and a controller coupled to the plurality of semiconductor thermocouples, and wherein activating the cooling system comprises operating the controller to drive the semiconductor thermocouples to extract heat from the transmitter assembly.
